(19) United States
(12) Patent Application Publication
(10) Pub. No.: US 2025/0325214 A1
(43) Pub. Date: Oct. 23, 2025

(54) NEUROFEEDBACK DEVICE WITH ELASTIC ELECTROENCEPHALOGRAPHY CAP

(71) Applicant: ExeBrain Co., Ltd., Taipei City (TW)

(72) Inventors: Shu-Chun Kuan, Taipei City (TW); Jason Chun-Cheng Lin, Taipei City (TW)

(21) Appl. No.: 18/964,571

(22) Filed: Dec. 1, 2024

(30) Foreign Application Priority Data
Apr. 19, 2024 (TW) 113114727

(51) Int. Cl. A61B 5/291; A61B 5/00; A61B 5/256; A61B 5/273

(52) U.S. Cl. CPC A61B 5/291; A61B 5/256; A61B 5/273; A61B 5/6814; A61B 2560/0443

(57) ABSTRACT

Provided is a neurofeedback device with elastic electroencephalography (EEG) cap, including: a cap body, a knob base, an electrode device, and a signal amplifier. The cap body is made of elastic material and includes a circuit layer and a buffer material. The buffer material covers the circuit layer elastically to ensure a wearer's comfort. The knob base passes through the cap and is electrically connected to the circuit layer. The electrode device includes a knob shell, a buckle cover and an electrode head. The knob shell has an inner space, and an outer surface with an external thread for threading to an internal threaded hole of the knob base to allow the electrode device to rotate for adjusting the wear tightness. The signal amplifier is used to receive electroencephalogram signals sensed by the electrode device, amplifies, analyzes, and transmits the signals to external devices.

### CROSS-REFERENCE TO RELATED APPLICATION

This application claims the priority of Taiwanese patent application No. 113114727, filed on Apr. 19, 2024, which is incorporated herewith by reference.

### BACKGROUND OF THE INVENTION

### 1. Field of the Invention

The present invention relates generally to an electroencephalography cap, and more particularly, to a neurofeedback device with elastic electroencephalography cap that can adapt to a wider range of wearers' head circumferences and facilitate the removal and assembly of electrodes.

### 2. The Prior Arts

With the development of biomedicine, there are more and more scientific methods based on analyzing biological signals, such as electrocardiogram, electromyogram or electroencephalogram. The brain is the main cognitive processing organ of the human body and responds to most external stimuli. Analyzing the brain's electroencephalogram can reveal how the human body processes and responds to external stimuli, as well as how brain wave will responses in different situations. Brain waves are essentially pyramidal neurons in the brain that are perpendicular to the cortical surface. The potential changes generated during activity can reflect how the brain behaves in response to external stimuli. Detecting brain waves is a non-invasive measurement. Detecting brain waves and analyzing the signals can effectively promote the research progress of human brain science.

It is known that electroencephalography caps will attach sensing electrodes to the subject's scalp to sense scalp potential changes when the subject is stimulated by the outside world. Brainwave signals are collected through sensing electrodes, amplifiers are used to increase the amplitude of the brainwave signals, and finally brainwave signals detected by sensing electrodes at different positions are combined for brainwave analysis. Common methods of using sensing electrodes include dry electrodes directly attached to the scalp, or wet electrodes using conductive paste as a medium between the electrodes and the scalp to reduce the resistance when measuring potential to improve measurement accuracy. When setting the sensing electrode positions, usually the sensing electrode device of the electroencephalography cap will use the 10-20 electrode calibration method to set each electrode position to measure the potential signal respectively. The 10-20 electrode calibration method employs five dividing lines to divide the head circumference between the skull and the occipital bone into six segments. The first and sixth segments account for 10% of the total distance and the rest are 20%, and then set the sensing electrodes at the intersections of each dividing line, forming 19 sensing electrode setting points, plus two ear sensing electrodes, to make a total of 21 electrode positions.

As the position of the sensing electrode will affect the accuracy and reliability of the electroencephalogram, it often takes much time to correct the position of the electrodes to avoid errors and poor contact. Therefore, there are electroencephalogram caps that install electrodes on the headgear and use adjustment devices to adapt to the head shapes of different subjects. However, given that the 10-20 electrode calibration method uses the ratio of the head circumference to set, the relative distance of each electrode will be different for different subjects, and it is difficult to adapt to various head circumferences using the adjustment device. If the tester's head circumference is larger or smaller than the common range, the adjustment device cannot be used to adjust to correct positions.

Patent No. M643701 of the Taiwan discloses an electroencephalogram device with a hard structure. Although the disclosure shows an adjustment device to adjust the electrodes, the adjustment range is still limited.

It is known that electroencephalography caps are hard structures and the adjustable range is mostly limited. The general head circumference of adults is between 50-62 cm. When worn by children and teenagers (<50 cm) or subjects with slightly larger head circumferences (>62 cm), each electrode setting point cannot contact the corresponding position of the subject scalp well. Moreover, because the electrode device of the conventional electroencephalography cap is designed as a kit, the electrodes cannot be easily disassembled and replaced. The electroencephalography cap must be removed from the subject's head before the electrodes can be disassembled and assembled, which increases adjustment work time; moreover, it is also impossible to adjust the tightness of a single electrode head to measure brainwave data more accurately. In addition, each electrode has its own wire connected to the data collection or amplification device, which is easy to get entangled with each other, making use more troublesome, and also restricts the subject's activity space.

It is imperative to address the shortcomings of conventional electroencephalography caps, such as the inability to adapt to the floating head of the wearer, the trouble of disassembling and assembling the electrode device, the inability to adjust the tightness of the contact between a single electrode and the head, and the trouble of winding the wire structure.

### SUMMARY OF THE INVENTION

A primary objective of the present invention is to provide a neurofeedback device with elastic electroencephalography cap, which is designed with elastic material and a knob structure to enable the electroencephalography cap to adapt to a wider range of head sizes of various subjects.

Another objective of the present invention is that the knob structure can be used to change different types of electrodes without removing the electroencephalogram cap from the subject's head, or to adjust the contact between the electrodes and the subject's scalp according to various needs. The tightness improves the measurement results and also makes it easier to disassemble and assemble the electrodes.

Another objective of the present invention is that the cap has a circuit layer for electrically connecting the electrode device, amplifier and analysis device to avoid wire connections to reduce entanglement or limit the wearer's mobility.

The neurofeedback device with elastic electroencephalography cap provided by the present invention includes: a cap body, a plurality of knob bases, a plurality of electrode devices, and an amplifier, wherein the side of the cap body contacting a subject's scalp is defined as the inner side, and the other side is defined as the outer side; the cap body is made of elastic material and includes: a circuit layer and buffer material; the circuit layer is made of conductive elastic material, electrically connected to each knob base, and the relative distance between the knob bases is configured according to the 10-20 electrode calibration method, which can be elongated equally when worn; the buffer material is an elastic insulating material covering the circuit layer to maintain wearer's comfort; the plurality of knob bases penetrates and connects the inner and outer sides of the cap body, and each knob base has an internal threaded hole with the internal threads only provided on corresponding inner peripheral surface of the outside of the knob base contacting the circuit layer; the electrode device includes: a knob shell, a buckle cover and an electrode; the knob shell is made of conductive material and has an internal accommodation space, and outer surface of the knob shell has external threads to be screwed together with the internal thread holes of the knob base, and inner surface has internal threads of the knob shell; the electrode is made of conductive material and is arranged in the inner space of the knob shell to sense potential changes contacting the subject's scalp, and has external threads of the electrode to screw into the internal threads of the knob shell; the buckle cover is fastened to the knob shell to fix the electrode; the amplifier receives signals sensed by the electrode device and amplifies or analyzes the signals before transmitting the signals to external devices.

Preferably, after the electrode device is installed on the knob base, the cap body is put on the subject's head, and the relative position of the electrode device is fixed by the elasticity of the cap body, so that the setting can be quickly completed. The electrode device senses the surface potential changes of the subject's scalp and transmits the signal to the amplifier to form an electroencephalogram.

Preferably, when the subject wears the electroencephalography cap, the electrode can be directly replaced, such as a wet electrode, a semi-wet electrode or a dry electrode, without disassembling the electroencephalography cap. Specifically, the operator uses the knob structure design to directly rotate and take out the electrode head from the outside of the electroencephalography cap while wearing it on the subject's head for replacement or maintenance.

In a preferred embodiment, the electrode can be further subdivided into a first-type electrode and a second-type electrode, wherein the first-type electrode is a semi-wet electrode tip, made of materials that can absorb conductive solutions, such as sponge, soft fiber or spherical fiber. It is also equipped with a replacement cartridge containing conductive solution, which can be installed into the first-type electrode to allow the conductive solution to leak out to reduce the resistance between the electrode and the scalp. After using the conductive solution, the buckle cover can be opened quickly to replace the cartridge to keep the electrode moist. The second-type electrode is a dry electrode, which has a brush head made of conductive material. The free end of the brush head has different forms, such as oblique, zigzag, and cylindrical shapes.

In a preferred embodiment, the amplifier is disposed on the cap body, and the electrode device transmits and processes brainwave signals through the amplifier and the circuit layer.

In a preferred embodiment, the amplifier and the cap body are provided separately from each other. The electrode device also has a wireless transmission unit. The electrode device transmits and processes brainwave signals through the wireless transmission unit, the circuit layer and the amplifier.

Preferably, the wireless transmission unit is Bluetooth wireless transmission unit.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a schematic perspective view of an embodiment of the present invention;

FIG. 2 is a side cross-sectional view of an embodiment of the present invention;

FIG. 3 is a cross-sectional view of the assembled structure of the electrode device and the cap body according to the embodiment of the present invention;

FIG. 4 is a cross-sectional view of the electrode device and the cap body disassembled according to the embodiment of the present invention;

FIG. 5 is a disassembled cross-sectional view of the electrode device according to the first embodiment of the present invention; and

FIG. 6 is a disassembled sectional view of the electrode device according to the second embodiment of the present invention.

### DETAILED DESCRIPTION OF THE PREFERRED EMBODIMENT

The technical solutions of the present invention will be described clearly and completely below in conjunction with the specific embodiments and the accompanying drawings. It should be noted that when an element is referred to as being “mounted or fixed to” another element, it means that the element can be directly on the other element or an intervening element may also be present. When an element is referred to as being “connected” to another element, it means that the element can be directly connected to the other element or intervening elements may also be present. In the illustrated embodiment, the directions indicated up, down, left, right, front and back, etc. are relative, and are used to explain that the structures and movements of the various components in this case are relative. These representations are appropriate when the components are in the positions shown in the figures. However, if the description of the positions of elements changes, it is believed that these representations will change accordingly.

Unless otherwise defined, all technical and scientific terms used herein have the same meaning as commonly understood by one of ordinary skill in the art of the present invention. The terminology used herein is for the purpose of describing particular embodiments only and is not intended to limit the present invention. As used herein, the term “and/or” includes any and all combinations of one or more of the associated listed items.

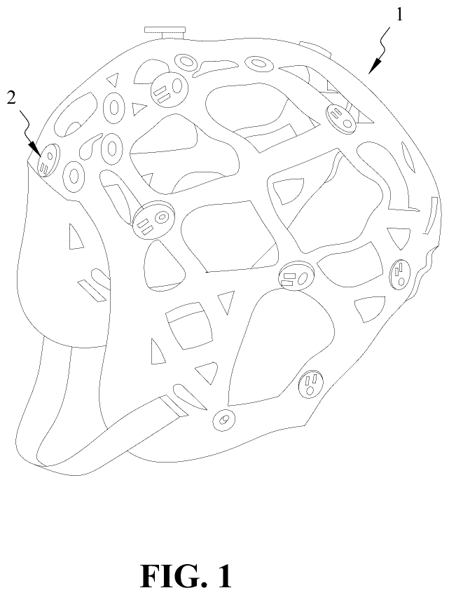

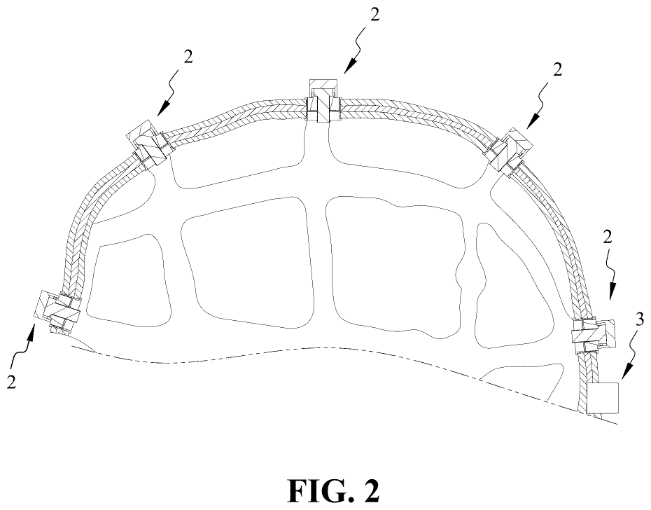

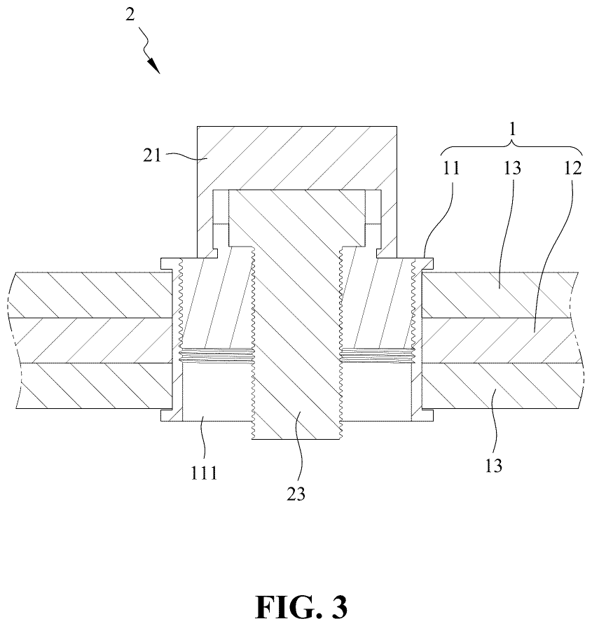

FIGS. 1-6 are schematic views of a neurofeedback device with elastic electroencephalography cap according to an embodiment of the present invention. FIG. 2 is a cross-sectional view of an embodiment of the present invention and FIG. 3 is a cross-sectional view after assembly of the electrode device and the cap body. The neurofeedback device with elastic electroencephalography cap of the present invention includes: a cap body 1, an electrode device 2, and an amplifier 3. The cap body 1 is made of elastic material, where the side in contact with the subject's scalp is defined as the inner side, and the other side is the outer side. The cap body 1 includes: a knob base 11, a circuit layer 12, and a buffer material 13. The knob base 11 penetrates the inner and outer sides of the cap body 1 and is electrically connected to the circuit layer 12. The knob base 11 has an internal threaded hole 111. The knob base 11 is proportionally arranged at corresponding sensing points on the cap body 1 according to the 10-20 electrode calibration method. The circuit layer 12 is made of elastic conductive material, and the circuit layer 12 has circuits that can electrically connect the amplifier 3 and each knob base 11 to transmit brainwave signals. The buffer material 13 is an elastic insulating material covering the circuit layer 12, which can be used as a pad to prevent the subject from discomfort and provide insulation.

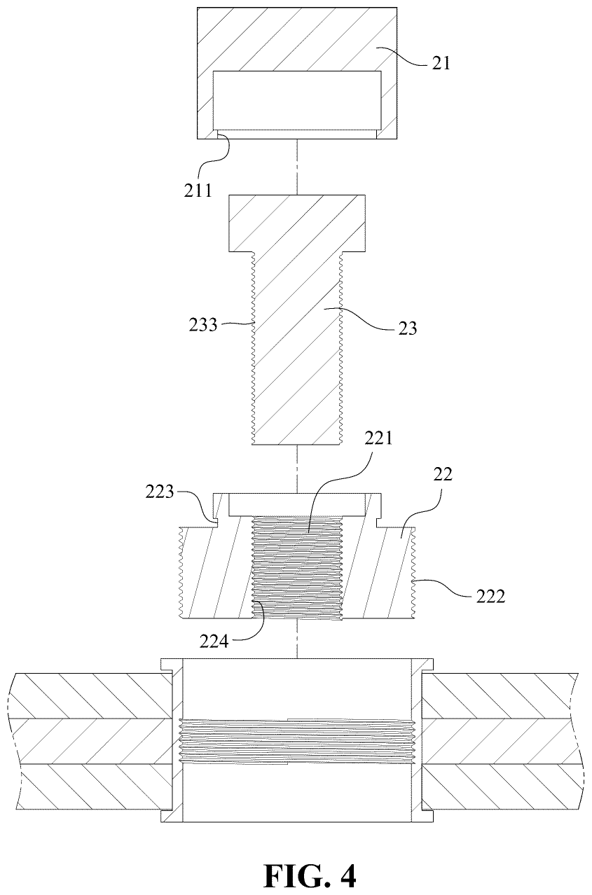

As shown in the cross-sectional view of the embodiment of the present invention in FIG. 2 and the cross-sectional view of the electrode device 2 and the cap body 1 disassembled in FIG. 4, the electrode device 2 includes: a buckle cover 21, a knob shell 22 and an electrode 23. The knob shell 22 has an inner accommodation space 221, a shell external thread 222 on the outer surface, and a shell internal thread 224 on the inner surface; the shell external thread 222 can be screwed together with the internal thread hole 111 of the knob base 11, so that the depth of entry of the knob shell 22 into the knob base 11 can be adjusted by rotation of the knob shell so as to control the tightness of the contact between the electrode 23 and the subject's scalp, as well as to be removed from the knob base 11. The electrode 23 can be placed into the inner accommodation space 221 from the outside of the knob shell 22, and protrudes from the inner side surface of the cap body 1 to contact the subject's scalp, electrically connect thereto and measure potential changes to generate an electroencephalogram. The outer surface of the electrode 23 has an electrode external thread 233, which is used to screw the shell internal thread 224 to adjust the tightness of the contact between the electrode 23 and the subject's scalp. The buckle cover 21 and the knob shell 22 is a paired tenon design, namely an outer latch 211 at the end of the buckle cover 21 close to the electroencephalogram cap and an inner latch 223 at the end of the knob shell 22 away from the electroencephalogram cap. After the electrode 23 is installed into the inner accommodation space 221, the outer latch 211 of the buckle cover 21 engages the inner latch 223 of the knob shell 22 from the outside to fix the electrode 23 to avoid loosening.

In a preferred embodiment of the present invention, after the electrode device 2 is installed on the knob base 11, the cap body 1 is put on the subject's head. By using the cap body 1 and the knob base 11 with a preset relative distance, the relative position of each electrode device 2 is quickly set and corrected on the subject's head. Therefore, the electrode device 2 can sense the potential changes in the subject's head and generate brainwave signals, which are sent to the amplifier 3 for analysis. Moreover, since the cap body 1 is made of elastic material, it can be adapted to a wider range of head circumferences of different wearers, and the elastic material is used to maintain the relative distance between the knob bases 11.

In a preferred embodiment of the present invention, the knob shell 22 can be removed from the knob base 11 when the cap is worn, and the electrode device 2 can be directly replaced without removing the cap body 1. As shown in FIG. 3, specifically, the internal threaded hole 111 only has threads on the inner side corresponding to the part where the outer surface of the knob base 11 is in contact with the circuit layer 12. The operator can unscrew the knob shell 22 to remove the electrode device 2 from the outside of the knob base 11 to replace or maintain the electrode device 2. As shown in FIG. 4, the operator can also directly rotate the electrode 23 out of the knob shell 22 and take out the electrode 23 from the outside of the knob shell 22. In summary, the present invention does not require removing the electroencephalogram cap from the wearer's head for disassembling and assembling the electrode device 2. The electrode device 2 can also be directly mounted back to the knob base 11 and then adjusted using the knob structural design.

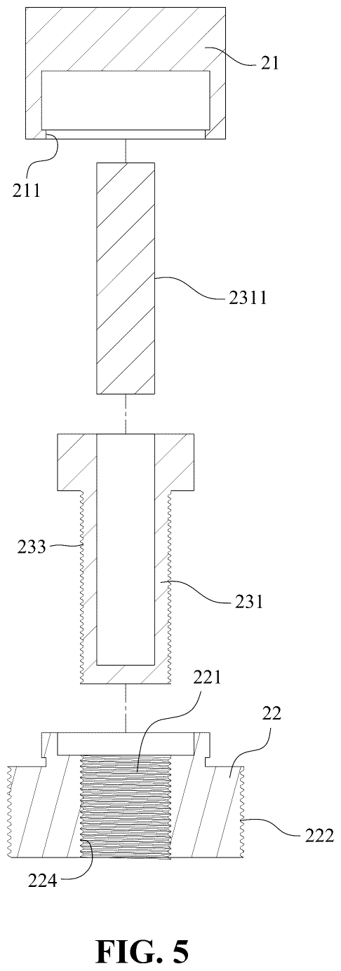

In a preferred embodiment of the present invention, the electrode 23 can be further subdivided into a first-type electrode 231 and a second-type electrode head 232. As shown in the disassembled cross-sectional view of the electrode device of the first embodiment of the present invention in FIG. 5, the first-type electrode 231 is a semi-wet electrode head, using a material that can absorb conductive solution as the electrode, such as sponge, soft fiber or spherical fibers. It is also equipped with a replacement cartridge 2311 installed in the first-type electrode 231 and filled with conductive solution. Replacing the cartridge 2311 allows the internal conductive solution to penetrate into the first-type electrode 231 to reduce the contact resistance between the electrode 23 and the subject's scalp and improve the sensitivity of sensing. After the conductive solution is exhausted, the electrode device 2 can be removed or the buckle cover 21 can be directly opened to replace the cartridge 2311 to keep the first-type electrode 231 moist.

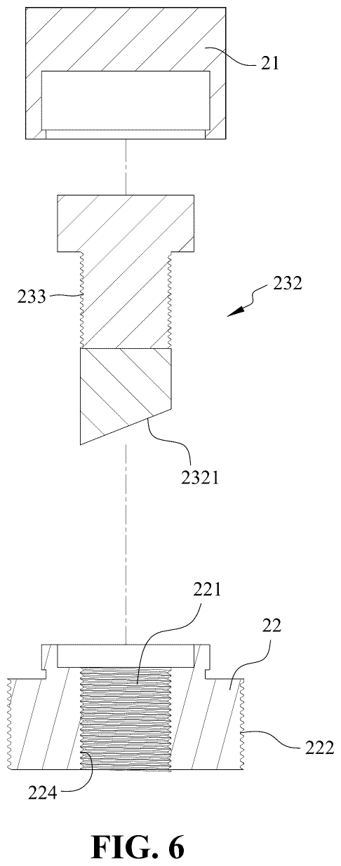

A preferred embodiment of the present invention, wherein, as shown in the disassembled cross-sectional view of the second embodiment of the present invention in FIG. 6, the second-type electrode 232 is a dry electrode head. The inner free end of close to the subject's scalp is equipped with a brush head 2321, which is used to contact the subject's scalp for electrical connection and measure potential changes. The brush head 2321 can be of different shapes such as oblique, zigzag, and cylindrical shapes.

In a preferred embodiment of the present invention, as shown in the side cross-sectional view of the electroencephalogram cap in FIG. 2, the amplifier 3 is disposed on the cap body 1, and the electrode device 2 transmits and processes the electroencephalogram signal through the amplifier 3 and the circuit layer 12.

In a preferred embodiment of the present invention, as shown in the side cross-sectional view of the electroencephalography cap in FIG. 2, the amplifier 3 and the cap body 1 are arranged separately from each other. The electrode device 2 also has a wireless transmission unit. The electrode device 2 transmits and analyzes brainwave signals through the wireless transmission unit, amplifier 3 and circuit layer 12.

Preferably, the wireless transmission unit can be of a Bluetooth wireless transmission technology.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A neurofeedback device with elastic electroencephalography cap, comprising:
a cap body, made of elastic material and comprising a circuit layer in the inside;
a plurality of knob bases, penetrating inside and outside of the cap body electrically connected to the circuit layer, each knob base having an internal thread hole;
a plurality of electrode devices, disposed inside the knob bases and able to be disassemble from outside of the cap body, each electrode device further comprising:
a knob shell, made of conductive material and forming an internal accommodation space, having shell external threads on outer surface for screwing together with the internal thread holes of the knob bases to adjust contact tightness between the electrode device and a subject's scalp, and having shell internal thread on inner surface;
an electrode, made of conductive material and arranged in the inner space of the knob shell to sense potential changes contacting the subject's scalp, and having electrode external threads to screw into the shell internal threads; and
a buckle cover, fastened to the knob shell from outside of the knob shell to fix the electrode;
an amplifier, for receiving signals sensed by the electrodes, processing and transmitting the signals.

2. The neurofeedback device with elastic electroencephalography cap according to claim 1, wherein the electrode comprises a first-type electrode and a replaceable cartridge disposed inside the first-type electrode, the cartridge contains a conductive solution able to penetrate the first-type electrode, and the first-type electrode is made of a material able to absorb the conductive solution and is used to contact the subject's scalp.

3. The neurofeedback device with elastic electroencephalography cap according to claim 1, wherein the electrode comprises a second-type electrode, having a brush head made of electrically conductive material for contacting the subject's scalp.

4. The neurofeedback device with elastic electroencephalography cap according to claim 3, wherein a free end of the brush head is of the oblique, zigzag, and cylindrical shapes

5. The neurofeedback device with elastic electroencephalography cap according to claim 2, wherein the cap body further comprises a buffer material covering the circuit layer and having properties of elasticity, ductility and insulation.

6. The neurofeedback device with elastic electroencephalography cap according to claim 5, wherein the amplifier is provided on the cap body, and the electrode device transmits and processes the brainwave signal through the amplifier and the circuit layer.

7. The neurofeedback device with elastic electroencephalography cap according to claim 5, wherein the amplifier and the cap body are provided separately from each other, and the electrode device also has a wireless transmission unit, and the electrode device transmits and processes the brainwave signal through the wireless unit, the amplifier and the circuit layer.

8. The neurofeedback device with elastic electroencephalography cap according to claim 7, wherein the wireless transmission unit is a Bluetooth wireless transmission unit.
